(19) United States
(12) Patent Application Publication
(10) Pub. No.: US 2023/0338661 A1
(43) Pub. Date: Oct. 26, 2023

(54) PRE-FILLED SYRINGE

(71) Applicant: NIPRO CORPORATION, Osaka-shi, Osaka (JP)

(72) Inventors: Ryo OGINO, Osaka-shi, Osaka (JP); Mitsuru HASEGAWA, Osaka-shi, Osaka (JP); Yuma SHIRAISHI, Osaka-shi, Osaka (JP)

(73) Assignee: NIPRO CORPORATION, Osaka-shi, Osaka (JP)

(21) Appl. No.: 18/025,698

(22) PCT Filed: Sep. 10, 2021
(86) PCT No.: PCT/JP2021/033288

§ 371 Date: Mar. 10, 2023

(30) Foreign Application Priority Data
Sep. 11, 2020 (JP) 2020-152937

(51) Int. Cl. A61M 5/28; A61M 5/19; A61M 5/315

(52) U.S. Cl. CPC A61M 5/28; A61M 5/19; A61M 5/31513

(57) ABSTRACT

A pre-filled syringe includes a tubular container, a front end gasket, a rear end gasket, an intermediate gasket, a medicine, a liquid medicine, and a plunger. The tubular container has a barrel and a nozzle portion. The barrel has an inner peripheral surface and a protruding inner side surface. A groove is formed in an outer peripheral surface of the intermediate gasket. The front end gasket has a rear-end pressure-contact portion. The nozzle portion has an accommodation portion. The accommodation portion has a bottom wall, a peripheral wall, and a supporting portion. The rear-end pressure-contact portion is in pressure contact with the inner peripheral surface of the barrel in an abutment state in which the liquid medicine is introduced in the front chamber and the front end portion of the front end gasket is in abutment with the supporting portion.

### TECHNICAL FIELD

The present invention relates to a pre-filled syringe.

### BACKGROUND ART

Conventionally, there has been known a pre-filled syringe in which a medicine and a liquid medicine can be mixed with each other in a container. For example, WO 2015/079874 discloses a pre-filled syringe including a tubular container, a front-end side gasket, a rear-end side gasket, an intermediate gasket, and a plunger. The tubular container has: a tubular portion that accommodates each gasket; and a nozzle portion attached to a front end of the tubular portion. The nozzle portion has an accommodation portion that can accommodate the front-end side gasket. An injection needle unit is attached to the front end of the nozzle portion. Each gasket is slidable in the tubular portion. A medicine is accommodated in a front chamber formed between the front-end side gasket and the intermediate gasket in the tubular portion, and a liquid agent is accommodated in a rear chamber formed between the rear-end side gasket and the intermediate gasket in the tubular portion. The plunger is connected to the rear-end side gasket and can push the rear-end side gasket toward the front end side.

The tubular portion has a bypass portion provided to protrude outward in the radial direction. A groove communicating the front chamber and the rear chamber with each other is formed in an outer peripheral surface of the intermediate gasket at a position at which the intermediate gasket faces the bypass portion.

In a state in which the plunger is not pushed, the intermediate gasket is located on the rear end side with respect to the bypass portion, and the front chamber and the rear chamber are partitioned by the intermediate gasket. When the plunger is pushed to cause the intermediate gasket to face the bypass portion, the front chamber and the rear chamber communicate with each other, with the result that the liquid agent accommodated in the rear chamber is pushed by the rear-end side gasket and is accordingly moved toward the front chamber via the groove portion of the intermediate gasket. Since the internal pressure of the front chamber is increased as the liquid agent is introduced in the front chamber, the front-end side gasket is accommodated in the accommodation portion. Thus, the accommodation portion and the front chamber communicate with each other, thereby unsealing the front chamber sealed by the front-end side gasket. Then, the plunger is pushed to a position at which the liquid level of the liquid agent does not reach the front end of the tubular portion, and the pre-filled syringe is shaken by a force to disperse the medicine. Thus, the medicine and the liquid agent are mixed with each other. When the plunger is further pushed from this state, the mixed liquid of the medicine and the liquid agent is discharged to the injection needle unit through a flow path formed between the front-end side gasket and the accommodation portion.

### CITATION LIST

### Patent Literature

PTL 1: WO 2015/079874

### SUMMARY OF INVENTION

### Technical Problem

In the pre-filled syringe described in WO 2015/079874, it is concerned that when the pre-filled syringe is shaken to mix the medicine and the liquid medicine with each other, part of the liquid agent reaches the accommodation portion through a clearance between the front-end side gasket and the tubular portion. When the part of the liquid agent reaches the accommodation portion during the shaking, part of the liquid agent may be leaked from the injection needle at the time of priming.

An object of the present invention is to provide a pre-filled syringe to suppress leakage of liquid from a front chamber during shaking.

### Solution to Problem

A pre-filled syringe according to one aspect of the present invention includes: a tubular container formed to have a tubular shape; a front end gasket provided in the tubular container, the front end gasket being slidable with respect to the tubular container along an axial direction of the tubular container; a rear end gasket provided rearward with respect to the front end gasket in the tubular container so as to define a space in the tubular container together with the front end gasket, the rear end gasket being slidable with respect to the tubular container along the axial direction of the tubular container; an intermediate gasket provided between the front end gasket and the rear end gasket in the tubular container so as to partition the space into a front chamber adjacent to the front end gasket and a rear chamber adjacent to the rear end gasket, the intermediate gasket being slidable with respect to the tubular container along the axial direction of the tubular container; a medicine accommodated in the front chamber; a liquid medicine accommodated in the rear chamber; and a plunger connected to the rear end gasket, the plunger being capable of pushing the rear end gasket toward a front end side of the tubular container along the axial direction, wherein the tubular container has a barrel capable of accommodating the front end gasket, the rear end gasket, and the intermediate gasket, and a nozzle portion connected to a front end portion of the barrel, the barrel has an inner peripheral surface in contact with an outer peripheral surface of the front end gasket, an outer peripheral surface of the intermediate gasket, and an outer peripheral surface of the rear end gasket, and a protruding inner side surface having a shape that protrudes from the inner peripheral surface in a direction orthogonal to the axial direction and that extends along the axial direction, a groove communicating the front chamber and the rear chamber with each other is found in the outer peripheral surface of the intermediate gasket at a position at which the intermediate gasket faces the protruding inner side surface, the front end gasket has a rear-end pressure-contact portion that is formed at a rear end portion of the front end gasket in the axial direction and that is in pressure contact with the inner peripheral surface of the barrel, the nozzle portion has an accommodation portion capable of accommodating the front end gasket, the accommodation portion has a bottom wall having a receiving surface that receives the front end gasket in the axial direction, a peripheral wall rising from an edge portion of the bottom wall, and a supporting portion provided on an inner peripheral surface of the peripheral wall at a region separated from the receiving surface in the axial direction, the supporting portion being capable of supporting a front end portion of the front end gasket, and the rear-end pressure-contact portion is in pressure contact with the inner peripheral surface of the barrel in an abutment state in which the liquid medicine is introduced in the front chamber and the front end portion of the front end gasket is in abutment with the supporting portion.

### Advantageous Effects of Invention

According to the present invention, there can be provided a pre-filled syringe to suppress leakage of liquid from a front chamber during shaking.

### BRIEF DESCRIPTION OF DRAWINGS

FIG. 1 is a perspective view showing a state before use of a pre-filled syringe according to one embodiment of the present invention.

FIG. 2 is a cross sectional view of the pre-filled syringe in the state before use.

FIG. 3 is a cross sectional view of a front end gasket.

FIG. 4 is a cross sectional perspective view of a vicinity of a nozzle portion.

FIG. 5 is a cross sectional view of a vicinity of a rib.

FIG. 6 is a cross sectional view of a vicinity of a front end gasket in an abutment state.

FIG. 7 is a cross sectional view of the pre-filled syringe in a pushing completion state.

### DESCRIPTION OF EMBODIMENTS

Embodiments of the present invention will be described with reference to figures. It should be noted that in the figures referred to below, the same or corresponding members are denoted by the same numerals.

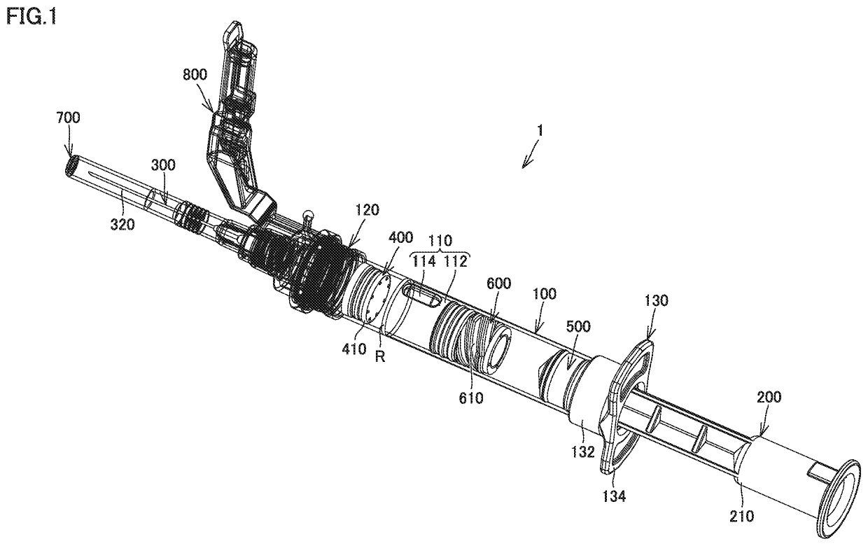

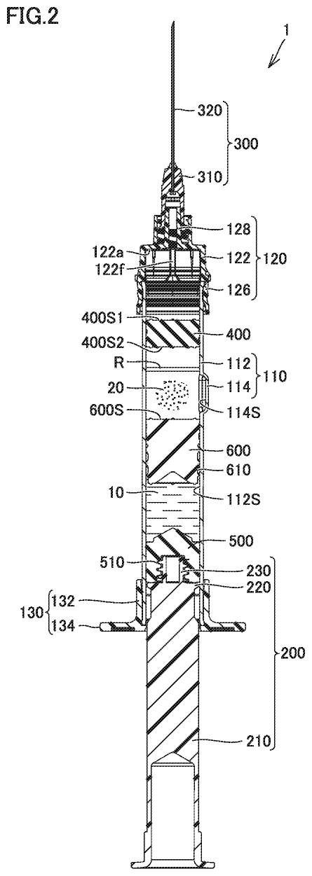

FIG. 1 is a perspective view of a pre-filled syringe according to one embodiment of the present invention. FIG. 2 is a cross sectional view of the pre-filled syringe. This pre-filled syringe 1 has a function as a container for a liquid medicine 10 and a medicine 20, and has a function as a syringe. As shown in FIGS. 1 and 2, pre-filled syringe 1 includes liquid medicine 10, medicine 20, a tubular container 100, a plunger 200, an injection needle unit 300, a front end gasket 400, a rear end gasket 500, an intermediate gasket 600, and a cap 700. It should be noted that each of FIGS. 1 and 2 shows pre-filled syringe 1 in a state before use.

Tubular container 100 accommodates liquid medicine 10 and medicine 20 with liquid medicine 10 and medicine 20 being separated from each other. That is, tubular container 100 has a function as a container for liquid medicine 10 and medicine 20. Tubular container 100 has a barrel 110, a nozzle portion 120, and a flange portion 130.

Barrel 110 accommodates liquid medicine 10 and medicine 20. As shown in FIG. 2, a front end portion (end portion on the upper side in FIG. 2) and a rear end portion (end portion on the lower side in FIG. 2) of barrel 110 are opened. Barrel 110 has a barrel main body 112 and a protrusion 114.

Barrel main body 112 is formed to have a tubular shape. Specifically, barrel main body 112 is formed to have a cylindrical shape.

Protrusion 114 has a shape that bulges from barrel main body 112 outward in a radial direction of barrel main body 112 and that extends along an axial direction (upward/downward direction in FIG. 2) of barrel main body 112. An inner side surface of protrusion 114 constitutes a protruding inner side surface 114S (see FIG. 2). Protruding inner side surface 114S has a shape that protrudes from an inner peripheral surface 112S of barrel main body 112 in a direction orthogonal to the axial direction and that extends along the axial direction.

Barrel main body 112 is provided with a stop ring R. Stop ring R indicates a reference for an amount of pushing of plunger 200 into tubular container 100. Stop ring R is provided in barrel main body 112 at a region on the front end side with respect to protrusion 114.

Front end gasket 400 is provided in tubular container 100. Specifically, as shown in FIGS. 1 and 2, front end gasket 400 is accommodated in barrel main body 112 on the front end side with respect to protrusion 114 before the use of pre-filled syringe 1. Front end gasket 400 is in close contact with inner peripheral surface 112S of barrel main body 112. Front end gasket 400 is slidable with respect to barrel main body 112 and accommodation portion 122 along the axial direction. In the present embodiment, the length of front end gasket 400 in the axial direction is set to 7.0 mm. The diameter of a front end surface 400S1 of front end gasket 400 is set to 14.45 mm.

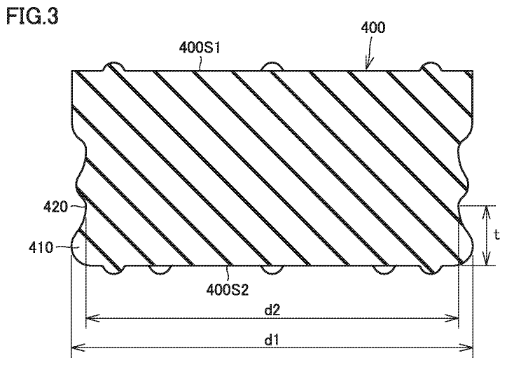

As shown in FIG. 3, front end gasket 400 is provided with a rear-end pressure-contact portion 410 and a recess 420.

Rear-end pressure-contact portion 410 is formed at a rear end portion of front end gasket 400. Rear-end pressure-contact portion 410 is in pressure contact with inner peripheral surface 112S of barrel main body 112. A rear end of rear-end pressure-contact portion 410 is connected to a rear end surface 400S2 of front end gasket 400. Rear end surface 400S2 is formed to be flat. Rear-end pressure-contact portion 410 has a shape that is curved to protrude outward in the radial direction (leftward/rightward direction in FIG. 3) and that is connected annularly to rear-end pressure-contact portion 410 itself in the peripheral direction. An outer diameter d1 (see FIG. 3) of a top portion of rear-end pressure-contact portion 410 is set to 14.45 mm.

Recess 420 is connected to rear-end pressure-contact portion 410. Recess 420 has a shape that is curved to protrude inward in the radial direction and that is connected annularly to recess 420 itself in the peripheral direction. An outer diameter d2 (see FIG. 3) of a bottom portion of recess 420 is set to 13.4 mm. A thickness t (see FIG. 3) of a region from rear end surface 400S2 to the bottom portion of recess 420 is set to 2.1 mm.

Rear end gasket 500 is provided rearward with respect to front end gasket 400 in tubular container 100 so as to define a space in tubular container 100 together with front end gasket 400. Rear end gasket 500 is in close contact with inner peripheral surface 112S of barrel main body 112. Rear end gasket 500 is slidable with respect to barrel main body 112 along the axial direction. As shown in FIG. 2, a female thread portion 510 is formed in rear end gasket 500.

Intermediate gasket 600 is provided between front end gasket 400 and rear end gasket 500 in barrel main body 112 of tubular container 100 so as to partition the space into a front chamber and a rear chamber. As shown in FIG. 2, intermediate gasket 600 is accommodated in barrel main body 112 on the rear end side with respect to protrusion 114 before the use of pre-filled syringe 1.

The front chamber is a space adjacent to front end gasket 400. More specifically, the front chamber is a space defined by front end gasket 400, intermediate gasket 600, and barrel 110. Medicine 20 is accommodated in the front chamber.

The rear chamber is a space adjacent to rear end gasket 500. More specifically, the rear chamber is a space defined by rear end gasket 500, intermediate gasket 600, and barrel 110. Liquid medicine 10 is accommodated in the rear chamber.

Intermediate gasket 600 is in close contact with inner peripheral surface 112S of barrel main body 112. Intermediate gasket 600 is slidable with respect to barrel main body 112 along the axial direction. A groove 610 communicating the front chamber and the rear chamber with each other is formed in the outer peripheral surface of intermediate gasket 600 at a position at which intermediate gasket 600 faces protruding inner side surface 114S.

Nozzle portion 120 is connected to the front end portion of barrel main body 112. Nozzle portion 120 has an accommodation portion 122, an attachment portion 126, and a discharge portion 128.

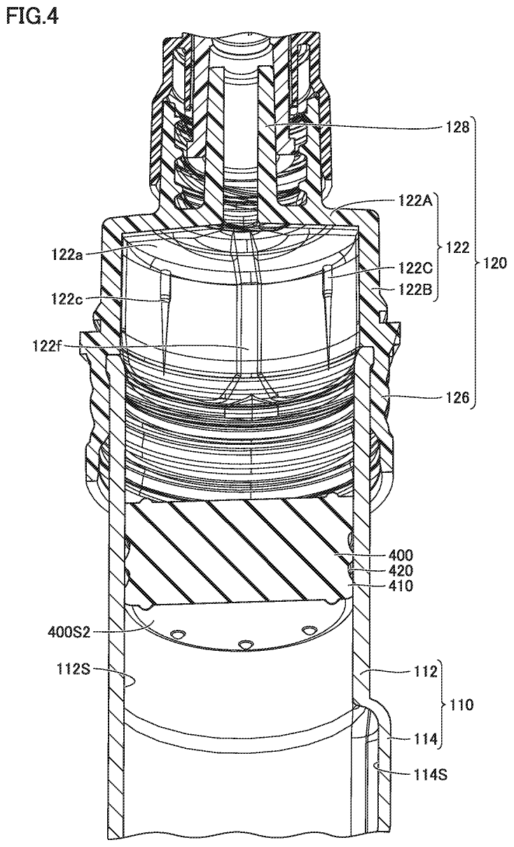

Accommodation portion 122 can accommodate front end gasket 400. Accommodation portion 122 is located on the front end side with respect to barrel main body 112. As shown in FIG. 1, a protective member 800 is attached to accommodation portion 122. As shown in FIG. 4, accommodation portion 122 has a bottom wall 122A, a peripheral wall 122B, and ribs 122C.

Bottom wall 122A has a receiving surface 122a that receives the front end portion of front end gasket 400 in the axial direction.

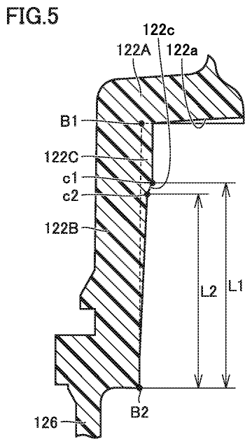

Peripheral wall 122B rises from an edge portion of bottom wall 122A. Peripheral wall 122B is formed to have a cylindrical shape. The inner diameter of a front end B1 (see FIG. 5) of peripheral wall 122B is smaller than the inner diameter of a rear end B2 (see FIG. 5) of peripheral wall 122B. In the present embodiment, the inner diameter of front end B1 is set to 13.5 mm, and the inner diameter of rear end B2 is set to 14.3 mm.

Each of ribs 122C is provided on an inner peripheral surface of peripheral wall 122B. Rib 122C has a shape that protrudes inward from the inner peripheral surface of peripheral wall 122B in the radial direction of peripheral wall 122B and that extends along the axial direction. The front end of rib 122C is connected to receiving surface 122a of bottom wall 122A. Ribs 122C are provided at equal intervals along the peripheral direction of peripheral wall 122B.

As shown in FIGS. 4 and 5, each of ribs 122C has a supporting portion 122c. Supporting portion 122c is a region that can support the front end portion of front end gasket 400. Supporting portion 122c is formed at an intermediate portion of rib 122C in the axial direction. That is, supporting portion 122c is provided on the inner peripheral surface of peripheral wall 122B at a region separated from receiving surface 122a in the axial direction. Supporting portion 122c is constituted of a stepped portion protruding inward in the radial direction of peripheral wall 122B from a region of rib 122C located on the rear side with respect to supporting portion 122c. As shown in FIG. 5, supporting portion 122c has a shape with its diameter being gradually decreased toward the front end in the axial direction.

A distance L1 (see FIG. 5) between rear end B2 of peripheral wall 122B and front end portion c1 of supporting portion 122c is set to 6.2 mm. A distance L2 (see FIG. 5) between rear end B2 of peripheral wall 122B and rear end portion c2 of supporting portion 122c is set to 5.9 mm. The inner diameter of front end portion c1 of supporting portion 122c is set to 13.9 mm. The inner diameter of rear end portion c2 of supporting portion 122c is set to 14 mm.

Rear end portion c2 of supporting portion 122c has a shape that is curved to protrude outward in the radial direction of peripheral wall 122B. In the present embodiment, the radius of curvature of rear end portion c2 is set to 0.7 mm.

In the inner peripheral surface of peripheral wall 122B and receiving surface 122a of bottom wall 122A, a flow path 122f for discharging a mixed liquid of liquid medicine 10 and medicine 20 is formed. This flow path 122f has a shape depressed from other regions of the inner peripheral surface of peripheral wall 122B and receiving surface 122a than flow path 122f. Flow path 122f is provided in the inner peripheral surface of peripheral wall 122B at a region different from the region in which rib 122C is provided.

Attachment portion 126 is a region that is attached to the front end portion of barrel main body 112. Attachment portion 126 has a shape extending rearward from a rear end portion of accommodation portion 122. Attachment portion 126 is formed to have a cylindrical shape.

Discharge portion 128 has a shape extending frontward from the front end of bottom wall 122A. The inside of discharge portion 128 is connected to flow path 122f.

Injection needle unit 300 is connected to discharge portion 128. Injection needle unit 300 has: a base body 310 connected to discharge portion 128; and a piercing needle 320 held by base body 310. Before pre-filled syringe 1 is used as a syringe, piercing needle 320 is covered with cap 700 (see FIG. 1).

Flange portion 130 is connected to a rear end portion of barrel main body 112. Flange portion 130 has an attachment tubular portion 132 and a projection 134.

Attachment tubular portion 132 is a region that is attached to the rear end portion of barrel main body 112. Attachment tubular portion 132 is formed to have a cylindrical shape.

Projection 134 has a shape projecting outward from the rear end portion of attachment tubular portion 132 in the radial direction.

Plunger 200 is connected to rear end gasket 500. Plunger 200 can push rear end gasket 500 toward the front end side of tubular container 100 along the axial direction. As shown in FIG. 2, plunger 200 has a stem portion 210, a pushing portion 220, and a connection portion 230.

Connection portion 230 is connected to rear end gasket 500. Connection portion 230 has a male thread portion screwed into female thread portion 510 of rear end gasket 500.

Pushing portion 220 is connected to a rear end portion of connection portion 230. Pushing portion 220 pushes rear end gasket 500. Pushing portion 220 is formed to have a circular plate shape.

Stem portion 210 has a shape extending rearward from the rear end surface of pushing portion 220 along the axial direction of barrel 110.

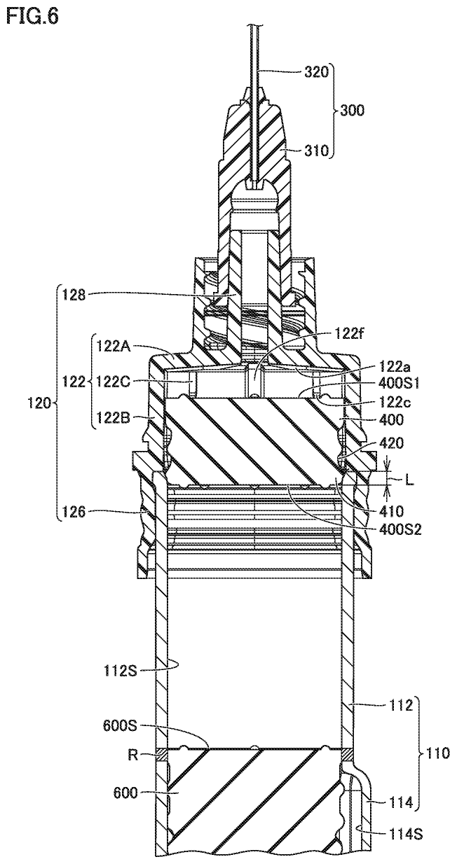

FIG. 6 is a cross sectional view of the vicinity of front end gasket 400 of pre-filled syringe 1 in an abutment state in which liquid medicine 10 is introduced in the front chamber and the front end portion (front end surface 400S1) of front end gasket 400 is in abutment with supporting portion 122c. It should be noted that in the abutment state, the front end portion of intermediate gasket 600 is located at the position of stop ring R.

As shown in FIG. 6, in the abutment state, rear-end pressure-contact portion 410 of front end gasket 400 is in pressure contact with inner peripheral surface 112S of barrel 110. An amount of overlap L (see FIG. 6) between front end gasket 400 and barrel 110 in the abutment state is set to an amount by which liquid medicine 10 in the front chamber is regulated from reaching accommodation portion 122 through a clearance between rear-end pressure-contact portion 410 and barrel 110 and gas (for example, air) in the front chamber is permitted to reach accommodation portion 122 through the clearance between rear-end pressure-contact portion 410 and barrel 110 in such a posture that the front end portion of barrel 110 faces upward. Amount of overlap L is preferably set to 0.5 mm or more and 2.2 mm or less. In the present embodiment, amount of overlap L is set to 1.1 mm.

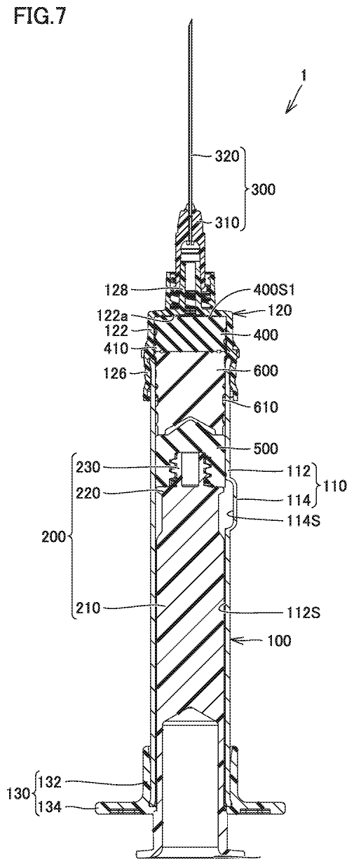

FIG. 7 is a cross sectional view in a pushing completion state (state in which plunger 200 is pushed into tubular container 100 until front end gasket 400 is brought into contact with receiving surface 122a, intermediate gasket 600 is brought into contact with front end gasket 400, and rear end gasket 500 is brought into contact with intermediate gasket 600).

As shown in FIG. 7, in the state in which front end surface 400S1 of front end gasket 400 is in contact with receiving surface 122a of accommodation portion 122, front end gasket 400 is separated from barrel main body 112. In this state, the mixed liquid is discharged through flow path 122f.

Next, a method of using pre-filled syringe 1 will be described.

First, as shown in FIG. 2, the posture of pre-filled syringe 1 is adjusted such that injection needle unit 300 faces upward. In this state, plunger 200 is pushed upward into tubular container 100.

When groove 610 of intermediate gasket 600 faces protruding inner side surface 114S, the front chamber and the rear chamber communicate with each other, with the result that liquid medicine 10 in the rear chamber is pushed by rear end gasket 500 to move toward the front chamber via the clearance between groove 610 of intermediate gasket 600 and protruding inner side surface 114S. On this occasion, as shown in FIG. 6, plunger 200 is pushed until front end surface 600S of intermediate gasket 600 coincides with stop ring R. Since the internal pressure of the front chamber is increased due to the pushing of plunger 200, front end gasket 400 is moved in the axial direction until front end gasket 400 is brought into abutment with supporting portion 122c of rib 122C (until the abutment state is attained).

Then, in the abutment state in which front end surface 400S1 of front end gasket 400 is in abutment with supporting portion 122c, rear-end pressure-contact portion 410 of front end gasket 400 is in pressure contact with inner peripheral surface 112S of barrel main body 112 as shown in FIG. 6. In this abutment state, pre-filled syringe 1 is shaken to mix liquid medicine 10 and medicine 20 with each other. Thus, liquid medicine 10 and medicine 20 are mixed with each other.

Part of liquid medicine 10 may be moved toward rear end surface 400S2 of front end gasket 400 during the shaking; however, in the present embodiment, rear-end pressure-contact portion 410 of front end gasket 400 is in pressure contact with inner peripheral surface 112S of barrel main body 112, with the result that leakage of liquid from the front chamber to accommodation portion 122 is effectively suppressed. On the other hand, during the shaking, gas in the front chamber exits into accommodation portion 122 through the clearance between rear-end pressure-contact portion 410 and barrel main body 112.

Thereafter, plunger 200 is further pushed. Accordingly, front end gasket 400 is separated from barrel main body 112, and front end surface 400S1 of front end gasket 400 is brought into abutment with receiving surface 122a. When plunger 200 is further pushed from this state, the mixed liquid is discharged through flow path 122f and piercing needle 320. Then, when the pushing completion state shown in FIG. 7 is attained, the discharging of the mixed liquid is ended.

As described above, in this pre-filled syringe 1, rear-end pressure-contact portion 410 of front end gasket 400 is in pressure contact with inner peripheral surface 112S of barrel 110 in the abutment state, thereby effectively suppressing leakage of liquid from the front chamber to accommodation portion 122 when pre-filled syringe 1 is shaken to mix medicine 20 and liquid medicine 10 with each other.

[Implementations]

It will be appreciated by those skilled in the art that the exemplary embodiments described above are specific examples of the following implementations.

A pre-filled syringe according to one aspect of the present disclosure includes: a tubular container formed to have a tubular shape; a front end gasket provided in the tubular container, the front end gasket being slidable with respect to the tubular container along an axial direction of the tubular container; a rear end gasket provided rearward with respect to the front end gasket in the tubular container so as to define a space in the tubular container together with the front end gasket, the rear end gasket being slidable with respect to the tubular container along the axial direction of the tubular container; an intermediate gasket provided between the front end gasket and the rear end gasket in the tubular container so as to partition the space into a front chamber adjacent to the front end gasket and a rear chamber adjacent to the rear end gasket, the intermediate gasket being slidable with respect to the tubular container along the axial direction of the tubular container; a medicine accommodated in the front chamber; a liquid medicine accommodated in the rear chamber; and a plunger connected to the rear end gasket, the plunger being capable of pushing the rear end gasket toward a front end side of the tubular container along the axial direction, wherein the tubular container has a barrel capable of accommodating the front end gasket, the rear end gasket, and the intermediate gasket, and a nozzle portion connected to a front end portion of the barrel, the barrel has an inner peripheral surface in contact with an outer peripheral surface of the front end gasket, an outer peripheral surface of the intermediate gasket, and an outer peripheral surface of the rear end gasket, and a protruding inner side surface having a shape that protrudes from the inner peripheral surface in a direction orthogonal to the axial direction and that extends along the axial direction, a groove communicating the front chamber and the rear chamber with each other is formed in the outer peripheral surface of the intermediate gasket at a position at which the intermediate gasket faces the protruding inner side surface, the front end gasket has a rear-end pressure-contact portion that is formed at a rear end portion of the front end gasket in the axial direction and that is in pressure contact with the inner peripheral surface of the barrel, the nozzle portion has an accommodation portion capable of accommodating the front end gasket, the accommodation portion has a bottom wall having a receiving surface that receives the front end gasket in the axial direction, a peripheral wall rising from an edge portion of the bottom wall, and a supporting portion provided on an inner peripheral surface of the peripheral wall at a region separated from the receiving surface in the axial direction, the supporting portion being capable of supporting a front end portion of the front end gasket, and the rear-end pressure-contact portion is in pressure contact with the inner peripheral surface of the barrel in an abutment state in which the liquid medicine is introduced in the front chamber and the front end portion of the front end gasket is in abutment with the supporting portion.

In this pre-filled syringe, since the rear-end pressure-contact portion of the front end gasket is in pressure contact with the inner peripheral surface of the barrel in the abutment state in which the liquid medicine is introduced in the front chamber and the front end portion of the front end gasket is in abutment with the supporting portion of the accommodation portion, leakage of liquid from the front chamber to the accommodation portion is effectively suppressed when the pre-filled syringe is shaken to mix the medicine and the liquid medicine with each other.

Further, preferably, an amount of overlap between the rear-end pressure-contact portion and the barrel in the abutment state is set to an amount by which the liquid medicine in the front chamber is regulated from reaching the accommodation portion through a clearance between the rear-end pressure-contact portion and the barrel and gas in the front chamber is permitted to reach the accommodation portion through the clearance between the rear-end pressure-contact portion and the barrel in such a posture that the front end portion of the barrel faces upward.

This makes it possible to attain both suppression of leakage of liquid from the front chamber to the accommodation portion when the pre-filled syringe is shaken in the abutment state and the effective exiting of gas from the front chamber at the time of priming.

Further, preferably, the front end gasket is provided with a recess connected to the rear-end pressure-contact portion, and the recess has a shape that is curved to protrude inward in a radial direction of the barrel and that is connected annularly to the recess itself in a direction of the barrel.

This makes it possible to facilitate exiting of the gas from the front chamber into the accommodation portion through the clearance between the rear-end pressure-contact portion and the barrel when the pre-filled syringe is shaken in the abutment state.

It should be noted that the embodiments disclosed herein are illustrative and non-restrictive in any respect. The scope of the present invention is defined by the terms of the claims, rather than the embodiments described above, and is intended to include any modifications within the scope and meaning equivalent to the terms of the claims.

### REFERENCE SIGNS LIST

1: pre-filled syringe; 10: liquid medicine; 20: medicine; 100: tubular container; 110: barrel; 112: barrel main body; 112S: inner peripheral surface; 114: protrusion; 114S: protruding inner side surface; 120: nozzle portion; 122: accommodation portion; 122A: bottom wall; 122a: receiving surface; 122B: peripheral wall; 122C: rib; 122c: supporting portion; 122f: flow path; 126: attachment portion; 128: discharge portion; 130: flange portion; 132: attachment tubular portion; 134: projection; 200: plunger; 210: stem portion; 220: pushing portion; 230: connection portion; 300: injection needle unit; 310: base body; 320: piercing needle; 400: front end gasket; 410: rear-end pressure-contact portion; 420: recess; 500: rear end gasket; 510: female thread portion; 600: intermediate gasket; 610: groove; 700: cap; 800: protective member.

1. A pre-filled syringe comprising:
a tubular container formed to have a tubular shape;
a front end gasket provided in the tubular container, the front end gasket being slidable with respect to the tubular container along an axial direction of the tubular container;
a rear end gasket provided rearward with respect to the front end gasket in the tubular container so as to define a space in the tubular container together with the front end gasket, the rear end gasket being slidable with respect to the tubular container along the axial direction of the tubular container;
an intermediate gasket provided between the front end gasket and the rear end gasket in the tubular container so as to partition the space into a front chamber adjacent to the front end gasket and a rear chamber adjacent to the rear end gasket, the intermediate gasket being slidable with respect to the tubular container along the axial direction of the tubular container;
a medicine accommodated in the front chamber;
a liquid medicine accommodated in the rear chamber; and
a plunger connected to the rear end gasket, the plunger being capable of pushing the rear end gasket toward a front end side of the tubular container along the axial direction, wherein
the tubular container has
a barrel capable of accommodating the front end gasket, the rear end gasket, and the intermediate gasket, and
a nozzle portion connected to a front end portion of the barrel,
the barrel has
an inner peripheral surface in contact with an outer peripheral surface of the front end gasket, an outer peripheral surface of the intermediate gasket, and an outer peripheral surface of the rear end gasket, and
a protruding inner side surface having a shape that protrudes from the inner peripheral surface in a direction orthogonal to the axial direction and that extends along the axial direction,
a groove communicating the front chamber and the rear chamber with each other is formed in the outer peripheral surface of the intermediate gasket at a position at which the intermediate gasket faces the protruding inner side surface,
the front end gasket has a rear-end pressure-contact portion that is formed at a rear end portion of the front end gasket in the axial direction and that is in pressure contact with the inner peripheral surface of the barrel,
the nozzle portion has an accommodation portion capable of accommodating the front end gasket,
the accommodation portion has
a bottom wall having a receiving surface that receives the front end gasket in the axial direction,
a peripheral wall rising from an edge portion of the bottom wall, and
a supporting portion provided on an inner peripheral surface of the peripheral wall at a region separated from the receiving surface in the axial direction, the supporting portion being capable of supporting a front end portion of the front end gasket, and
the rear-end pressure-contact portion is in pressure contact with the inner peripheral surface of the barrel in an abutment state in which the liquid medicine is introduced in the front chamber and the front end portion of the front end gasket is in abutment with the supporting portion.

2. The pre-filled syringe according to claim 1, wherein an amount of overlap between the rear-end pressure-contact portion and the barrel in the abutment state is set to an amount by which the liquid medicine in the front chamber is regulated from reaching the accommodation portion through a clearance between the rear-end pressure-contact portion and the barrel and gas in the front chamber is permitted to reach the accommodation portion through the clearance between the rear-end pressure-contact portion and the barrel in such a posture that the front end portion of the barrel faces upward.

3. The pre-filled syringe according to claim 1, wherein
the front end gasket is provided with a recess connected to the rear-end pressure-contact portion, and
the recess has a shape that is curved to protrude inward in a radial direction of the barrel and that is connected annularly to the recess itself in a peripheral direction of the barrel.

4. The pre-filled syringe according to claim 2, wherein
the front end gasket is provided with a recess connected to the rear-end pressure-contact portion, and
the recess has a shape that is curved to protrude inward in a radial direction of the barrel and that is connected annularly to the recess itself in a peripheral direction of the barrel.
